(19) United States
(12) Patent Application Publication
(10) Pub. No.: US 2013/0189358 A1
(43) Pub. Date: Jul. 25, 2013

(54) SAXAGLIPTIN PHARMACEUTICAL FORMULATIONS

(71) Applicants: Roey SOLOMONOVICH, Tel Aviv (IL); Dafna ARIELI, Kadima (IL)

(72) Inventors: Roey SOLOMONOVICH, Tel Aviv (IL); Dafna ARIELI, Kadima (IL)

(21) Appl. No.: 13/738,332

(22) Filed: Jan. 10, 2013

Related U.S. Application Data
(60) Provisional application No. 61/584,921, filed on Jan. 10, 2012.

(51) Int. Cl. A61K 9/28; A61K 9/20; A61K 31/403

(52) U.S. Cl. CPC A61K 9/284; A61K 31/403; A61K 9/205; A61K 9/2013; A61K 9/2018; A61K 9/2095; A61K 9/2893
USPC 424/465; 514/412

(57) ABSTRACT

The present invention includes a compressed solid dosage form comprising saxagliptin or a pharmaceutically acceptable salt thereof and at least one pharmaceutically acceptable excipient, wherein the at least one pharmaceutically acceptable excipient optionally comprises at least one organic acid.

### CROSS REFERENCE OF PRIORITY APPLICATION

The present patent application claims the benefit of U.S. Provisional Application, No. 61/584,921, filed on 10 Jan. 2012, the disclosures of which are herein incorporated by reference.

### BACKGROUND OF THE INVENTION

The present invention relates to formulations of dipeptidyl peptidase IV (DPP4) inhibitors. In particular, the present invention relates to formulations of a class of cyano-pyrrolidine-based DPP4 inhibitors, such as saxagliptin and processes for their preparation.

Saxagliptin, (1S,3S,5S)-2-((2S)-2-amino-2-(3-hydroxyadamantan-1-yl)-acetyl)-2-azabicyclo[3.1.0]hexane-3-carbonitrile, belongs to a class of cyano-pyrrolidine-based DPP4 inhibitors, and has the following chemical formula:

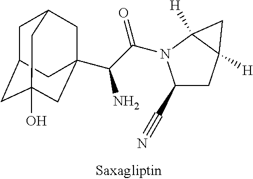 [SMILES: N#C[C@@H]1C[C@@H]2C[C@@H]2N1C(=O)[C@@H](N)C12CC3CC(CC(O)(C3)C1)C2]

Saxagliptin, in the form of its hydrochloride salt, is marketed under the trade name ONGLYZA® by Bristol-Myers Squibb for the treatment of type 2 diabetes mellitus. Each film-coated tablet of ONGLYZA for oral use contains either 2.79 mg saxagliptin hydrochloride (anhydrous) equivalent to 2.5 mg saxagliptin, or 5.58 mg saxagliptin hydrochloride (anhydrous) equivalent to 5 mg saxagliptin and the following inactive ingredients: lactose monohydrate, microcrystalline cellulose, croscarmellose sodium, and magnesium stearate. In addition, the film coating contains the following inactive ingredients: polyvinyl alcohol, polyethylene glycol, titanium dioxide, talc, and iron oxides.

It is known that saxagliptin has a propensity to chemical instability due to intramolecular cyclization of the compound by attack of the amino group on the nitrile group, to form a cyclic amidine impurity:

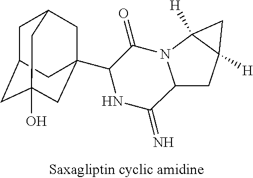 [SMILES: N=C1NC(C23CC4CC(CC(O)(C4)C2)C3)C(=O)N2C1C[C@@H]1C[C@@H]12]

This cyclic amidine impurity is therapeutically inactive.

G. Scott Jones et al, J. Org. Chem., Kinetic and Mechanistic Insight into the Thermodynamic Degradation of Saxagliptin, Nov. 4, 2011 (Web), describes the undesirable conversion of saxagliptin to amidine and subsequently to the diketopiperazine byproduct, which in the present invention are referred to as cyclic amidine (“AMD”) and oxamidine (“OXAMD”) respectively. The hydrolysis of amidine to diketopiperazine occurs in the presence of water.

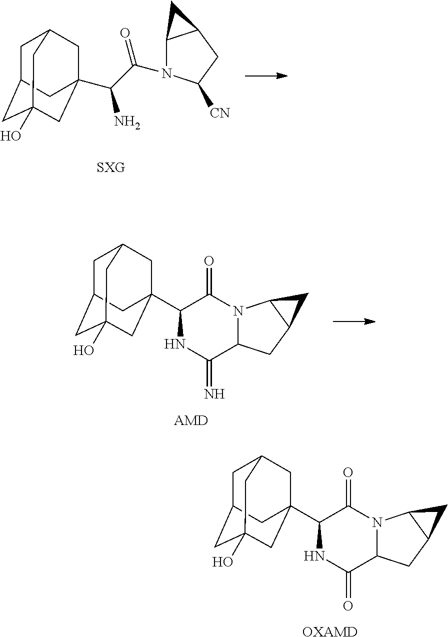

WO 2005/117841 discloses that the intramolecular cyclization reaction to form the cyclic amidine can occur both in the solid state and in the solution state, and moreover can be exacerbated by the use of processing conditions such as wet granulation, roller compaction or tabletting. WO 2005/117841 further discloses that commonly used excipients can accelerate the rate of this intramolecular cyclization reaction.

Clearly, this chemical instability results in the need to provide conditions and excipients that either minimize or avoid this undesirable reaction from occurring during manufacture of saxagliptin formulations. The challenge of minimizing or preventing the cyclization reaction during manufacture of saxagliptin formulations is particularly significant because saxagliptin is a low dose drug, which is typically administered in dosage forms containing 2.5 or 5 mg of the drug, and hence the ratio of excipients to drug is high. Thus, a further challenge exists in the selection of compatible excipients that do not cause or accelerate the cyclization reaction.

In a purported attempt to provide a suitable formulation of saxagliptin, WO2005/117841 and US7951400 disclose a process involving coating the drug onto an inert tablet core. The inert tablet core is first provided an inner seal coat layer onto which the drug layer is applied. A further layer is preferably provided as a protecting coating. The layers are preferably applied by spray coating. The inner seal coat is typically comprised of a coating polymer such as polyvinyl alcohol. Thus, by providing a dosage form wherein the drug is applied as a layer or coating, and wherein the dosage form (and in particular, the drug component) is not subjected to compression forces, the potential for formation of the cyclic amidine due to the application of a compression force to prepare the dosage form is avoided.

However, the process disclosed in WO2005/117841 is cumbersome since it involves the need to provide the various layers as solutions or suspensions in an organic or aqueous solvent with subsequent drying steps after the application of each layer. The use of organic solvents, which although may be easier to remove in the drying step after application of the coating, is undesirable. However, the use of aqueous solvent typically requires a longer drying time, thus prolonging exposure of the labile drug substance to heat and moisture.

US2010/0074950 discloses anti-diabetic combination formulations containing a slow release biguanide, such as metformin, and a DPP4 inhibitor, such as saxagliptin. The formulations contain a tablet core containing the biguanide in a slow release form. The cores are then provided with a subcoat layer and a seal coating layer, upon which a layer containing the DPP4 inhibitor is applied.

Therefore, there exists a need to provide more simple formulations and processes for preparing formulations of DPP4 inhibitors such as saxagliptin that have a propensity to degrade via intramolecular cyclization.

The present invention provides formulations of such DPP4 inhibitors, which can be readily prepared by simple formulation processes and which do not suffer from the potential complications and disadvantages of the prior art formulations.

### SUMMARY OF THE INVENTION

The present invention provides formulations of saxagliptin. In particular, according to one aspect of the present invention, there is provided a compressed solid dosage form comprising saxagliptin or a pharmaceutically acceptable salt thereof and at least one pharmaceutically acceptable excipient.

The dosage forms of the present invention can be prepared by compression of a mixture of saxagliptin (or a pharmaceutically acceptable salt thereof) and at least one pharmaceutically acceptable excipient.

Thus in a further aspect, the present invention provides a process for preparing the above dosage forms comprising the steps of: (a) providing a mixture of the saxagliptin or a pharmaceutically acceptable salt thereof and the at least one pharmaceutically acceptable excipient; and (b) compressing the mixture.

The invention further provides a dosage form obtainable by such a process.

According to another aspect, the present invention provides a pharmaceutical dosage form comprising saxagliptin or a pharmaceutically acceptable salt thereof and at least one solid pharmaceutically acceptable organic acid.

The present invention further provides the use of a solid pharmaceutically acceptable organic acid to stabilize a pharmaceutical dosage form comprising saxagliptin or a pharmaceutically acceptable salt thereof.

The solid dosage forms of the present invention avoid the problems and complications of the prior art layered compositions such as disclosed in WO2005/117841 and US7951400, in which the drug is provided as a layer (typically applied by spray coating) over an inert compressed core.

In particular, the solid dosage forms of the present invention are typically monolithic, hence, the saxagliptin is not applied as a separate layer in the dosage form. Thus, the dosage forms of the present invention can typically be prepared by compression of a mixture of saxagliptin or a pharmaceutically acceptable salt thereof and the at least one pharmaceutically acceptable excipient.

Although the dosage form of the present invention does not have a layered structure with respect to the drug, the dosage form may, nevertheless, contain an optional cosmetic coat.

The present invention further provides a process for preparing the dosage form comprising: (a) providing a mixture of the saxagliptin or a pharmaceutically acceptable salt thereof and the at least one pharmaceutically acceptable excipient; and (b) compressing the mixture.

The dosage forms of the present invention, being compressed are structurally stable and advantageously, the active agent does not undergo significant and unacceptable degradation to the cyclic amidine.

In a further aspect, the present invention provides a dosage form comprising saxagliptin or a pharmaceutically acceptable salt.

In a still further aspect, the present invention provides the use of a solid pharmaceutically acceptable organic acid to stabilize a pharmaceutical dosage form comprising saxagliptin or a pharmaceutically acceptable salt thereof.

### DETAILED DESCRIPTION OF THE INVENTION

The dosage forms of the present invention can typically be prepared by compression of a mixture of saxagliptin or a pharmaceutically acceptable salt thereof and the at least one pharmaceutically acceptable excipient. The saxagliptin or the pharmaceutically acceptable salt of the dosage forms of the present invention typically does not exist as a discrete, substantially uniform layer in the dosage forms.

As used herein, unless indicated otherwise, “room temperature” (“RT”) refers to typical ambient temperatures, i.e. temperatures in the range of about 15 to about 29° C., preferably about preferably about 20 to about 25° C., and more preferably about 25° C.

As used herein, unless indicated otherwise, percentages are given as wt % relative to the weight of the dosage form.

As used herein, unless indicated otherwise, the amount of saxagliptin cyclic amidine in the dosage forms following manufacture of the pharmaceutical dosage forms are measured following storage for 30 days at room temperature conditions (typically manufacture (following storage at room temperature (i.e. typically a temperature of between 15-29° C.), and at a relative humidity of not more than 67%.

As used herein, unless indicated otherwise, the organic acid is preferably a solid organic acid having a pKa1 of greater than 3. The organic acid may be defined by solubility in water. Unless indicate otherwise, preferably, the organic acid used herein has a solubility of 10 mg/ml or more in water at 20° C. to 25° C., preferably a solubility of 30 mg/ml or more in water at 20° C. to 25° C. and more preferably a solubility of 50 mg/ml or more in water at 20° C. to 25° C., such as 100 mg/ml or more in water at 20° C. to 25° C.

Alternatively, the organic acid is a solid organic acid having a pKa1 of 5 or less, and preferably no more than 5. In some of the embodiments, the organic acid has a pKa1 in the range of 2.5 to 5, e.g., in the range of 2.5 to no more than 5, preferably in the range of about 3 to less than 5. Examples of the organic acid having a pKa1 of about 3 include tartaric acid, fumaric acid and citric acid. The organic acid that can be used in the pharmaceutical dosage form comprising saxagliptin or a pharmaceutically acceptable salt thereof of the present invention include tartaric acid, fumaric acid, succinic acid, citric acid and ascorbic acid. The organic acid may be defined by solubility in water. Unless indicate otherwise, preferably, the organic acid used herein has a solubility of 10 mg/ml or more in water at 20° C. to 25° C., preferably a solubility of 30 mg/ml or more in water at 20° C. to 25° C. and more preferably a solubility of 50 mg/ml or more in water at 20° C. to 25° C., such as 100 mg/ml or more in water at 20° C. to 25° C.

The organic acid can also be a solid organic monocarboxylic acid having a pKa of 5 or less, and preferably no more than 5. In some of the embodiments, the organic acid has a pKa in the range of 2.5 to 5, e.g., in the range of 2.5 to no more than 5, preferably in the range of about 3 to less than 5. Alternatively, the organic acid is a solid organic monocarboxylic acid having a pKa of greater than 3. The organic acid may be defined by solubility in water. Unless indicate otherwise, preferably, the organic acid used herein has a solubility of 10 mg/ml or more in water at 20° C. to 25° C., preferably a solubility of 30 mg/ml or more in water at 20° C. to 25° C. and more preferably a solubility of 50 mg/ml or more in water at 20° C. to 25° C., such as 100 mg/ml or more in water at 20° C. to 25° C.

According to a first aspect of the present invention, there is provided a compressed solid dosage form comprising saxagliptin or a pharmaceutically acceptable salt thereof and at least one pharmaceutically acceptable excipient.

As used herein, the “compressed solid dosage form” of the present invention is a solid dosage form that can be prepared by direct compression of a mixture of the saxagliptin or a pharmaceutically acceptable salt thereof and at least one pharmaceutically acceptable excipient, or prepare by wet or dry granulation involving compression of a mixture of the saxagliptin or a pharmaceutically acceptable salt thereof and at least one pharmaceutically acceptable excipient.

Suitable pharmaceutically acceptable excipients in the dosage forms include those selected from the group consisting of filler, organic acid, disintegrant, lubricant, and optionally a binder, or a mixture of these excipients.

In one of the preferred embodiment of the present invention, the dosage forms comprise at least one pharmaceutically acceptable excipient selected from the group consisting of filler, organic acid, disintegrant, and lubricant, or a mixture thereof.

Preferably, the dosage forms of the present invention include at least one organic acid.

More preferably, the dosage forms of the present invention comprise saxagliptin or a pharmaceutically acceptable salt thereof and include at least a filler, organic acid, disintegrant and lubricant as pharmaceutically acceptable excipients.

Suitable fillers include those selected from the group consisting of microcrystalline cellulose, sorbitol, dextrose, sucrose, mannitol, dibasic calcium phosphate, starch, and mixtures thereof.

Preferred fillers include those selected from the group consisting of microcrystalline cellulose, sorbitol, dextrose, sucrose, mannitol, starch, and mixtures thereof. More preferably, the filler is selected from the group consisting of microcrystalline cellulose, mannitol, and mixtures thereof. Mannitol is a particularly preferred filler.

The filler can typically be present in an amount of about 60 to about 90 wt % of the dosage form. More preferably, the filler is present in an amount of about 65 to about 85 wt %, particularly about 70 to about 82 wt % of the dosage form. In especially preferred embodiments, the filler is present in an amount of about 72 to about 80 wt % of the dosage form.

The organic acid for use in the dosage forms of the present invention preferably have a pKa1 greater than 3. Preferably, the organic acid is a solid organic acid. The solid organic acid preferably has a water solubility at 20-25° C., of about 10 mg/ml or more, preferably about 30 mg/ml or more, more preferably about 50 mg/ml or more, and most preferably about 100 mg/ml or more.

Alternatively, the organic acid is a solid organic acid having a pKa1 of 5 or less, and preferably no more than 5. In some of the embodiments, the organic acid has a pKa1 in the range of 2.5 to 5, e.g., in the range of 2.5 to no more than 5, preferably in the range of about 3 to less than 5.

Suitable pharmaceutically acceptable acids include those selected from the group consisting of tartaric acid, fumaric acid, succinic acid, citric acid, ascorbic acid, and mixtures thereof. More preferably, the organic acid is selected from the group consisting of tartaric acid, succinic acid, citric acid, ascorbic acid, and mixtures thereof. Tartaric acid and citric acid are particularly preferred. In especially preferred embodiments, the organic acid is tartaric acid, preferably L-tartaric acid.

The organic acid is preferably employed in the dosage form in an excess relative to the weight of saxagliptin or pharmaceutically acceptable salt thereof in the dosage form. For example, the weight ratio of saxagliptin or pharmaceutically acceptable salt thereof to the organic acid can be in the range of from 1:5 to 1:12. Preferably, the weight ratio of saxagliptin or pharmaceutically acceptable salt thereof to the organic acid is in the range of from 1:6 to 1:10. More preferably, the weight ratio of saxagliptin or pharmaceutically acceptable salt thereof to the organic acid is in the range of from 1:5 to 1:8.

In the pharmaceutical dosage form comprising saxagliptin or a pharmaceutically acceptable salt thereof of the present invention, the organic acid can be present not in a weight excess relative to saxagliptin or the pharmaceutically acceptable salt. In some of the embodiments of the pharmaceutical dosage form comprising a pharmaceutically acceptable salt of saxagliptin, the organic acid can be present not in a weight excess relative to the pharmaceutically acceptable salt of saxagliptin. In some of the embodiments of the pharmaceutical dosage form of the present invention, the organic acid is present in a weight less than the weight of the saxagliptin or a pharmaceutically acceptable salt of saxagliptin.

The weight ratio of saxagliptin or pharmaceutically acceptable salt thereof to the organic acid can be in the range of from 1:1 to 10:1. Alternatively, the weight ratio of saxagliptin or pharmaceutically acceptable salt thereof to the organic acid is in the range of from 1:1 to 5:1. The weight ratio of saxagliptin or pharmaceutically acceptable salt thereof to the organic acid can also in the range of from 2:1 to 4:1.

Typically, in preferred embodiments of the present invention, the organic acid may be present in an amount of about 5 to about 25 wt % based on the total weight of the composition. Preferably, the organic acid is present in an amount of about 5 to about 15 wt % based on the total weight of the composition. More preferably, the organic acid is present in an amount of about 6 to about 12 wt % based on the total weight of the composition. In particularly preferred embodiments, the organic acid is present in an amount of about 8 to about 10 wt % based on the total weight of the composition.

Suitable disintegrants useful in the dosage forms of the present invention include those selected from the group consisting of croscarmellose sodium, alginic acid, microcrystalline cellulose, crospovidone, polacrilin potassium, sodium starch glycolate, low-substituted hydroxypropyl cellulose, starch, and mixtures thereof. Croscarmellose sodium, alginic acid, crospovidone, sodium starch glycolate, and mixtures thereof are preferred. In particularly preferred dosage forms of the present invention, the disintegrant is croscarmellose sodium.

The disintegrant may be present in the dosage form in an amount of about 4 to about 15 wt %, preferably about 5 to about 13 wt %, and more preferably about 7 to about 11 wt %.

Suitable lubricants for use in the dosage forms of the present invention include those selected from the group consisting of magnesium stearate, calcium stearate, glyceryl behenate, sodium stearyl fumarate, stearic acid, talc, zinc stearate, and mixtures thereof. Magnesium stearate, glyceryl behenate, sodium stearyl fumarate, talc and mixtures thereof are preferred. Particularly, the lubricant is selected from the group consisting of magnesium stearate, talc or mixtures thereof. Most preferably, the lubricant is magnesium stearate.

The lubricant is typically used in the dosage form in an amount of about 0.3% to about 3 wt %, preferably in an amount of about 0.5 to about 2 wt % and more preferably in an amount of about 0.4 to about 1.5 wt %.

In some embodiments of the present invention, the dosage form may include a binder. Suitable binders include those selected from the group consisting of polyvinyl pyrrolidone, hydroxypropyl cellulose, low-substituted hydroxypropyl cellulose, hydroxypropyl methyl cellulose, polyvinyl alcohol, starch and mixtures thereof. Polyvinyl pyrrolidone, hydroxypropyl cellulose, low-substituted hydroxypropyl cellulose, hydroxypropyl methyl cellulose, polyvinyl alcohol and mixtures thereof are particularly preferred. Hydroxypropyl methyl cellulose is an especially preferred binder.

Typically, the binder can be used in concentration of about 2 to about 6 wt %, and more preferably about 3 to about 5 wt %.

In preferred embodiments of the present invention, the saxagliptin or pharmaceutically acceptable salt thereof is present in an amount of about 1.0 to about 5 wt %, particularly about 1.2 to about 3.0 wt % and especially about 1.2 to about 2.3 wt %.

In preferred dosage forms of the present invention, the saxagliptin is preferably in the form of a pharmaceutically acceptable salt. The pharmaceutically acceptable salt of the saxagliptin may be prepared in advance, or may be prepared in situ during the formulation process, for example by contacting the saxagliptin base with the pharmaceutically acceptable acid during the formulation process. Preferred pharmaceutically acceptable salts of saxagliptin include those selected from the group consisting of hydrochloride, hydrobromide, phosphate or solvates and hydrates thereof. The hydrochloride or hydrobromide salts, or their solvates/hydrates thereof, are preferred. Saxagliptin hydrochloride salt, or its solvates and hydrates are especially preferred.

The present invention provides compressed dosage forms of saxagliptin that are structurally stable, and further, despite being subjected to compression, are stable with respect to the formation of saxagliptin cyclic amidine. In particular, the dosage forms of the present invention preferably contain less than 0.5 wt % of saxagliptin cyclic amidine 30 days after manufacture (following storage at ambient conditions (i.e. room temperature—i.e. a temperature of between 15-29° C.—and not more than 67% relative humidity).

Preferably, the dosage forms of the present invention contain 0.4 wt % or less, more preferably 0.35 wt %, and most preferably 0.3 wt % or less of saxagliptin cyclic amidine following storage for 30 days after manufacture at ambient conditions as described above.

In preferred embodiments of the present invention, the dosage forms comprise saxagliptin or a pharmaceutically acceptable salt thereof, preferably saxagliptin hydrochloride, and the following excipients: mannitol, tartaric acid, croscarmellose sodium, magnesium stearate, and optionally hydroxypropyl methylcellulose.

The dosage forms of the present invention are not layered. In particular, the active agent is present within the body of the dosage form, and is not present as a layer, in contrast to the prior art formulations. In particular, the dosage forms of the present invention are made by compressing a mixture containing saxagliptin and at least one pharmaceutically acceptable excipient as described below. However, the dosage forms may be optionally provided with a top coat which can include a cosmetic coat or an enteric coat. The top coat, however, does not contain saxagliptin or a pharmaceutically acceptable salt thereof.

The dosage forms of the present invention, being compressed, are structurally stable. Typically, the dosage forms have a hardness of 7 to 25 Strong-Cobb units (SCU), preferably a hardness of 7-20 SCU and most preferably a hardness of 8-18 SCU.

Dosage forms of the present invention may be prepared by a process comprising compressing a mixture of saxagliptin or a pharmaceutically acceptable salt thereof and a least one pharmaceutically acceptable excipient. The pharmaceutically acceptable excipient is preferably defined in any of the above-described embodiments. Preferably, the excipient comprises the following: mannitol, tartaric acid, croscarmellose sodium, magnesium stearate, and optionally hydroxypropyl methylcellulose. The saxagliptin or pharmaceutically acceptable salt thereof is preferably as defined in any of the above-described embodiments.

The dosage forms of the present invention, despite being formed by a process involving compression of the saxagliptin, are surprisingly stable to formation of saxagliptin cyclic amidine. Typically, the dosage forms of any embodiment of the present invention as herein described contain less than 0.5 wt %, particularly 0.4 wt % or less, more particularly 0.35 wt % or less and especially 0.3 wt % or less, of saxagliptin cyclic amidine or salts thereof following storage for 30 days following manufacture at ambient conditions of temperature and relative humidity (room temperature of between 15-29° C.), and not more than 67% relative humidity).

The present invention further provides a process for preparing the dosage forms as described in any of the above-mentioned embodiments. The process comprises the steps of:

(a) providing a mixture of the saxagliptin or a pharmaceutically acceptable salt thereof and the at least one pharmaceutically acceptable excipient; and

(b) compressing the mixture.

The mixture in step (a) may be prepared by wet granulation or dry granulation. If wet granulation is used, the mixture is subsequently subjected to a drying step to form a dried granulate, following which the dried granulate may be milled.

As indicated above, the pharmaceutical compositions of the present invention may comprise saxagliptin in the form of its free base, or preferably, in the form of a pharmaceutically acceptable salt thereof. In the preferred embodiments of the present invention, wherein the dosage form contains saxagliptin in the form of a pharmaceutically acceptable salt, the saxagliptin starting material for the formulation process may be provided as the pharmaceutically acceptable salt, or alternative as the free base. In the case of the latter, the formulation process includes a step whereby the saxagliptin base is contacted with a solution of a suitable pharmaceutically acceptable acid in order to form the corresponding pharmaceutically acceptable salt of saxagliptin. For example, the pharmaceutically acceptable acid may be employed as a solution which is contacted with the saxagliptin free base. The solution can be employed, e.g. as a granulation medium for wet granulation. Thus, in the embodiments of the process of the present invention, where wet granulation is employed, the saxagliptin starting material may be in the form of the free base, which is contacted with a solution of a pharmaceutically acceptable acid to form the corresponding pharmaceutically acceptable salt thereof.

As indicated above, the preferred pharmaceutically acceptable salts of saxagliptin include hydrochloride, hydrobromide and phosphate, or solvates and hydrates thereof. Hence, solutions (preferably aqueous) of the corresponding acids—namely hydrochloric acid, hydrobromic acid, or phosphoric acid—may be employed as the wet granulation medium to form the corresponding salt of saxagliptin. In more preferred embodiments, the saxagliptin is in the form of its hydrochloride, and thus, the saxagliptin base is contacted with hydrochloric acid during the formulation process (e.g. by employing hydrochloric acid as the wet granulation solution).

In step (b) the mixture of step (a) is compressed to form tablets. The tablets may be coated with a top coat which can be a cosmetic coating or an enteric coating or other extended release coating.

The cosmetic coat can be any suitable protective coating, and may include a coating polymer, preferably selected from the group consisting of polyvinyl pyrrolidone, hydroxypropyl cellulose, hydroxypropyl methyl cellulose, polyvinyl alcohol and mixtures thereof, and preferably wherein the coating polymer is selected from the group consisting of polyvinyl pyrrolidone, polyvinyl alcohol or a mixture thereof, and more preferably polyvinyl alcohol. Suitable cosmetic coatings include commercially available systems such as Opadry® coatings (Colorcon).

The present invention further provides a dosage form obtainable by the above-described process.

The present invention further provides a pharmaceutical dosage form comprising saxagliptin or a pharmaceutically acceptable salt thereof and at least one solid pharmaceutically acceptable organic acid. The dosage form is preferably a compressed mixture of saxagliptin or a pharmaceutically acceptable salt thereof, and the at least one solid pharmaceutically acceptable organic acid. The organic acid preferably has a pKa1 greater than 3.

Alternatively, the organic acid is a solid organic acid having a pKa1 of 5 or less, and preferably no more than 5. In some of the embodiments, the organic acid has a pKa1 in the range of 2.5 to 5, e.g., in the range of 2.5 to no more than 5, preferably in the range of about 3 to less than 5.

The organic acid preferably has a solubility in water at 20-25° C. of about 10 mg/ml or more, preferably about 30 mg/ml or more, more preferably about 50 mg/ml or more and most preferably about 100 mg/ml or more.

Suitable organic acids include those selected from the group consisting of tartaric acid, fumaric acid, succinic acid, citric acid, ascorbic acid, and mixtures thereof. Tartaric acid, citric acid, and mixtures thereof are preferred. More preferably, the organic acid is tartaric acid, particularly L-tartaric acid.

The organic acid is preferably present in an excess relative to the saxagliptin or pharmaceutically acceptable salt thereof. Typically, the weight ratio of saxagliptin or pharmaceutically acceptable salt thereof to the organic acid is in the range of from 1:5 to 1:12, preferably from 1:6 to 1:10, and more preferably in the range of from 1:5 to 1:8. In preferred embodiments of this aspect of the present invention, the organic acid may be used in an amount of about 5 to about 25 wt %, preferably about 5 to about 15 wt %, and more preferably about 6 to about 12 wt % based on the total weight of the composition. Particularly, the organic acid is used in an amount of about 8 to about 10 wt % based on the total weight of the composition.

In this aspect of the present invention, the dosage form preferably further comprises at least one pharmaceutically acceptable excipient. Suitable excipients include those selected from the group consisting of filler, disintegrant, lubricant, and optionally a binder, or a mixture thereof. Preferably, the pharmaceutically acceptable excipient includes at least a filler, disintegrant and lubricant.

Suitable and preferred fillers, disintegrants, lubricants, and binders are described above.

The saxagliptin or pharmaceutically acceptable salt thereof is preferably present in an amount of about 1.0 to about 5 wt %, preferably about 1.2 to about 3.0 wt %, and more preferably about 1.2 to about 2.3 wt %. Preferably the saxagliptin is in the form of a pharmaceutically acceptable salt, such as hydrochloride, hydrobromide, phosphate or solvates and hydrates thereof. Preferably, the saxagliptin is in the form of its hydrochloride or hydrobromide salt, or solvates and hydrates thereof. More preferably, the saxagliptin is in the form of its hydrochloride.

In a particularly preferred embodiment of this aspect of the invention, the dosage form comprises saxagliptin or a pharmaceutically acceptable salt thereof (preferably saxagliptin hydrochloride), and the following excipients: mannitol, tartaric acid, croscarmellose sodium, magnesium stearate, and optionally hydroxypropyl methylcellulose.

In a further aspect, the present invention further comprises the use of a solid pharmaceutically acceptable organic acid to stabilize a pharmaceutical dosage form comprising saxagliptin or a pharmaceutically acceptable salt thereof. Preferably the pharmaceutically acceptable organic acid is a solid at room temperature. Preferably, the organic acid has a pKa1 greater than 3. The organic acid preferably has a solubility in water at 20-25° C. of 10 mg/ml or more, preferably 30 mg/ml or more, more preferably 50 mg/ml or more and most preferably 100 mg/ml or more. Alternatively, the organic acid is a solid organic acid having a pKa1 of 5 or less, and preferably no more than 5. In some of the embodiments, the organic acid has a pKa1 in the range of 2.5 to 5, e.g., in the range of 2.5 to no more than 5, preferably in the range of about 3 to less than 5.

Suitable pharmaceutically acceptable organic acids include tartaric acid, fumaric acid, succinic acid, citric acid, ascorbic acid, and mixtures thereof. Tartaric acid, citric acid, and mixtures thereof are preferred. Most preferably, the organic acid is tartaric acid, preferably L-tartaric acid. The organic acid is preferably used in the dosage form an excess relative to the saxagliptin or pharmaceutically acceptable salt thereof. Preferably, the weight ratio of saxagliptin or pharmaceutically acceptable salt thereof to the organic acid is in the range of from 1:5 to 1:12, more preferably, 1:6 to 1:10, and most preferably from 1:5 to 1:8. The organic acid is typically used in an amount of about 5 to about 25 wt %, preferably about 5 to about 15 wt %, and more preferably about 6 to about 12 wt %, based on the total weight of the dosage form.

In the use aspect of the present invention, the pharmaceutical dosage form comprises saxagliptin or a pharmaceutically acceptable salt thereof and a pharmaceutically acceptable excipient. The excipient can include a filler, disintegrant, lubricant and optionally a binder, or a mixture thereof, as described above. Preferably, the dosage form comprises saxagliptin or a pharmaceutically acceptable salt thereof, and the following excipients: mannitol, tartaric acid, croscarmellose sodium, magnesium stearate, and optionally hydroxypropyl methylcellulose. The saxagliptin is preferably in the form of a pharmaceutically acceptable salt, preferably saxagliptin hydrochloride, or hydrates/solvates thereof.

In some of the embodiments of the solid pharmaceutical dosage form of the present invention comprising saxagliptin or a pharmaceutically acceptable salt thereof, the saxagliptin or the pharmaceutically acceptable salt thereof is present as particles having a particle size distribution wherein at least 50% of the particles are in the range of 260 micron to 500 micron, preferably at least 70% of the particles are in the range of 280 micron to 400 micron. For instance, in some of the embodiments of the compressed pharmaceutical tablet of the present invention, the saxagliptin or the pharmaceutically acceptable salt thereof is present as particles having a particle size distribution wherein at least 50% of the particles are in the range of 260 micron to 500 micron, preferably at least 70% of the particles are in the range of 280 micron to 400 micron.

The present invention is further illustrated by the following examples, which are not intended to limit the scope of the invention. It will be appreciated that various modifications are within the spirit and scope of the invention.

### EXAMPLES

Measurements of % wt impurities herein (oxamidine, cyclic amidine, etc) are taken 30 days after manufacture of the formulations [storage at ambient conditions, i.e. room temperature (RT) of between 15-29° C. and not more than 67% relative humidity (RH)].

### Example I

### Saxagliptin HCl Tablets formed by Direct Compression

Saxagliptin HCl, mannitol, tartaric acid and croscarmellose sodium (part I) were screened (Mesh#14) and mixed using a diffusion blender for 10 min. Magnesium stearate (part II) was then screened (mesh 50) and mixed with part I for an additional 3 min. The final blend was compressed into 8 mm round cores with a target average weight of 224.4 mg using a rotary press machine.

The process can be summarized in the following way:
1. De-lumping of agglomerates

### 2. Dry Mixing

3. Compression into Tablets

### Composition of Formulation #I

Cores

| | | |
|---|---|---|
| Raw Material Description | mg/TAB | %/TAB |

Part I - Mixing I

| | | |
|---|---|---|
| Saxagliptin HCl 1.25 H₂O | 2.995 | 1.4 |
| Mannitol (Pearlitol 200SD) | 180.0 | 79.8 |
| Tartaric acid (powder) | 20.00 | 9.1 |
| Croscarmellose Sodium (Ac-Di-Sol) | 20.00 | 9.1 |

Part II - Mixing II

| | | |
|---|---|---|
| Magnesium stearate | 1.40 | 0.6 |
| Total weight | 224.40 | 100% |

Formulation #1 Excluding Cosmetic Coating—Analytical Data (30 Days after Tablet Manufacture)

| | | | Greatest | |
|---|---|---|---|---|
| | | Cyclic | Unknown | Total |
| | Oxamidine | Amidine | Impurity | Impurity |
| Sample | level | level | level | level |
| Compressed core | <0.03% | 0.27% | 0.25% | 0.62% |
| High Hardness | | | | |
| (16.5 SCU) | | | | |
| 30 days at RT and | | | | |
| NMT 67% RH | | | | |
| Saxagliptin HCl | <0.03% | <0.03% | 0.19% | 0.23% |

### Example II

### Saxagliptin HCl Tablets Formed by Direct Compression (Prophetic)

Saxagliptin HCl, mannitol, tartaric acid and croscarmellose sodium (part I) are screened (Mesh#14) and mixed using a diffusion blender for 10 min. Magnesium stearate (part II) is then screened (mesh 50) and mixed with part I for additional 3 min. The final blend is then compressed into 8 mm round cores with an average target weight of 215 mg using a rotary press machine.

The compressed cores are finally coated by cosmetic opadry coating applying a pan coater machine. The opadry dispersion (part III) is prepared by first mixing opadry with water followed by drop wise addition of concentrated hydrochloric acid (36.5-38.0% w/w of HCl based on USP32-NF27 specification) in a titration manner to get a final pH of 2. The target weight for the coated tablet is 220 mg.

The process can be summarized in the following way:

1. De-lumping of agglomerates

### 2. Dry Mixing

3. Compression into Tablets

### 4. Cosmetic Coating

### Proposed Formulation #II

Cores

| | | |
|---|---|---|
| Raw Material Description | mg/TAB | %/TAB |

Part I - Mixing I

| | | |
|---|---|---|
| Saxagliptin HCl 1.25 H₂O | 2.995 | 1.4 |
| Mannitol (Pearlitol 200SD) | 170.61 | 77.5 |
| Tartaric acid (powder) | 20.00 | 9.1 |
| Croscarmellose Sodium (Ac-Di-Sol) | 20.00 | 9.1 |

Part II - Mixing II

| | | |
|---|---|---|
| Magnesium stearate | 1.40 | 0.6 |

Part III - Cosmetic coating

| | | |
|---|---|---|
| Opadry II Pink (Polyvinyl alcohol based) | 5.00 | 2.3 |
| pH 2.0 Acidic Medium* | 28.00 | |
| Total weight | 220.00 | 100% |

*Evaporated during process

### Example III

### Saxagliptin HCl Tablets Formed by Dry Granulation (Prophetic)

Saxagliptin HCl, mannitol, tartaric acid and croscarmellose sodium (part I) are screened (Mesh#14) and mixed using a diffusion blender for 10 min. Magnesium stearate (part II) is then screened (mesh 50) and mixed with part I for additional 3 min. The final blend is then compressed into 20 mm round slugs with an average target weight of 1500 mg applying a rotary press machine.

Alternatively, the final blend is compressed in ribbons applying a roller compaction machine.

The compressed slugs or ribbons are then milled using any of the three common types of milling machine (oscillating granulator/rotating impeller or hammer mill) with a suitable screen of 0.6-1.0 mm.

The milled granulate is then mixed with croscarmellose sodium (part III) in a diffusion blender for 5 min and finally magnesium stearate (part IV) is screened (mesh 50) and mixed in the diffusion blender for an additional period of 3 min.

The final blend is then compressed into 8 mm round cores with an average target weight of 215 mg applying a rotary press machine.

The compressed cores are finally coated by cosmetic (opadry) coating applying a pan coater machine. The opadry dispersion (part V) is prepared by first mixing opadry with water followed by drop wise addition of concentrated hydrochloric acid (36.5-38.0% w/w of HCl based on USP32-NF27 specification) in a titration manner to get a final pH of 2. The target weight for the coated tablet is 220 mg.

The process can be summarized in the following way:

1. De-lumping of agglomerates
2. Dry Mixing (part I)
3. Sieving part II
4. Dry mixing (part I+II)
5. Compression into Slugs or Ribbons

### 6. Milling

7. Mixing (part III)
8. Mixing (part IV)
9. Compression into Tablets

### 10. Cosmetic Coating

### Proposed Formulation #III

Cores

| | | |
|---|---|---|
| Raw Material Description | mg/TAB | %/TAB |

Part I - Mixing I

| | | |
|---|---|---|
| Saxagliptin HCl 1.25 H₂O | 2.995 | 1.4 |
| Mannitol (Pearlitol 200SD) | 179.51 | 77.5 |
| Tartaric acid (powder) | 20.00 | 9.1 |
| Croscarmellose Sodium(Ac-Di-Sol) | 10.00 | 9.1 |

Part I - Mixing II

| | | |
|---|---|---|
| Magnesium stearate | 1.50 | 0.7 |

Part III - Mixing III

| | | |
|---|---|---|
| Croscarmellose Sodium (Ac-Di-Sol) | 10.00 | 4.3 |

Part IV - Mixing IV

| | | |
|---|---|---|
| Magnesium stearate | 1.40 | 0.6 |

Part V - Cosmetic coating

| | | |
|---|---|---|
| Opadry II Pink (Polyvinyl alcohol based) | 5.00 | 2.2 |
| pH 2.0 Acidic Medium* | 28.00 | |
| Total weight | 220.00 | 100% |

*Evaporated during process

### Example IV

### Saxagliptin HCl Tablets Formed by Wet Granulation (Prophetic)

Saxagliptin HCl, mannitol, tartaric acid and croscarmellose sodium (part I) are screened (Mesh#14) and mixed using either a diffusion blender, a pneumatic blender (e.g. fluid-bed) or high-shear mixer. Wet-granulation is then performed by stepwise addition of 0.01 N hydrochloric acid (pH=2) to get a dense and pliable wet mass.

The wet granulation process can be done using either a high-shear mixer, a rotor granulator or top-spray granulator.

The wet mass is then dried at 40-60° C. using a fluid bed dryer until the final loss on drying (LOD) is less than 1%.

The dried mass is then milled using any of the three common types of milling machine (oscillating granulator/rotating impeller or hammer mill) with a suitable screen of 0.6-1.0 mm.

The milled granulate is then mixed with croscarmellose sodium (part III) in a diffusion blender for 5 min and finally magnesium stearate (part IV) is screened (mesh 50) and mixed in the diffusion blender for an additional period of 3 min. The final blend is then compressed into 8 mm round cores with an average weight of 225 mg applying a rotary press machine.

The compressed cores are finally coated by cosmetic (opadry) coating applying a pan coater machine. The opadry dispersion (part V) is prepared by first mixing opadry with water followed by drop wise addition of concentrated hydrochloric acid (36.5-38.0% w/w of HCl based on USP32-NF27 specification) in a titration manner to get a final pH of 2. The target weight for the coated tablet is 230 mg.

The process can be summarized in the following way:

1. De-lumping of agglomerates

### 2. Dry Mixing

3. Wet-granulation using high-shear/spray-granulation/rotor granulation

### 4. Drying

### 5. Milling

### 6. Mixing

7. Compression into Tablets

### 8. Cosmetic Coating

### Proposed Formulation #IV

Cores

| | | |
|---|---|---|
| Raw Material Description | mg/TAB | %/TAB |

Part I - Mixing I

| | | |
|---|---|---|
| Saxagliptin HCl 1.25 H₂O | 2.995 | 1.30 |
| Mannitol (Pearlitol 200SD) | 181.01 | 78.70 |
| Tartaric acid (powder) | 20.00 | 8.70 |
| Croscarmellose Sodium (Ac-Di-Sol) | 10.00 | 4.35 |

Part II - Wet granulation

| | |
|---|---|
| pH 2.0 Acidic medium* | 20.00 |

Part III - Mixing II

| | | |
|---|---|---|
| Croscarmellose Sodium (Ac-Di-Sol) | 10.00 | 4.35 |

Part IV - Mixing II

| | | |
|---|---|---|
| Magnesium stearate | 1.40 | 0.61 |

Part V - Cosmetic coating

| | | |
|---|---|---|
| Opadry II Pink (Polyvinyl alcohol based) | 5.00 | 2.17 |
| pH 2.0 Acidic Medium* | 28.00 | |
| Total weight | 230.00 | 100% |

*Evaporated during process

### Example V

### Saxagliptin HCl Tablets Formed by Wet Granulation (Prophetic)

Mannitol, tartaric acid and croscarmellose sodium (part I) are screened (Mesh#14) and mixed using either a diffusion blender, a pneumatic blender (e.g. fluid-bed) or high-shear mixer.

Granulation solution is prepared by dissolving saxagliptin free base in 0.01 N hydrochloric acid (pH=2) to which a binder is added. The granulation solution pH is then adjusted by addition of concentrated hydrochloric acid (36.5-38.0% w/w of HCl based on USP32-NF27 specification) in a titration manner to get a final pH of 2.

Wet-granulation is then performed by stepwise addition of the granulation solution to get a dense and pliable wet mass (additional Purified water is added if needed) The wet granulation process can be done using either a high-shear mixer, a rotor granulator or top-spray granulator.

The wet mass is then dried at 40-60° C. using a fluid bed dryer until the final loss on drying (LOD) is less than 1%. The dried mass is then milled using any of the three common types of milling machine (oscillating granulator/rotating impeller or hammer mill) with a suitable screen of 0.6-1.0 mm.

The milled granulate is then mixed with croscarmellose sodium (part III) in a diffusion blender for 5 min and finally magnesium stearate (part IV) is screened (mesh 50) and mixed in the diffusion blender for an additional period of 3 min. The final blend is then compressed into 8 mm round cores with an average weight of 225 mg applying a rotary press machine.

The compressed cores are finally coated by cosmetic (Opadry) coating applying a pan coater machine. The Opadry dispersion (part V) is prepared by first mixing Opadry with water followed by drop wise addition of concentrated hydrochloric acid (36.5-38.0% w/w of HCl based on USP32-NF27 specification) in a titration manner to get a final pH of 2. The target weight for the coated tablet is 230 mg.

The process can be summarized in the following way:

1. De-lumping of agglomerates

### 2. Dry Mixing

3. Wet-granulation using high-shear/spray-granulation/rotor granulation

### 4. Drying

### 5. Milling

### 6. Mixing

7. Compression into Tablets

### 8. Cosmetic Coating

### Proposed Formulation #V

Cores

| | | |
|---|---|---|
| Raw Material Description | mg/TAB | %/TAB |

Part I - Mixing I

| | | |
|---|---|---|
| Mannitol (Pearlitol 200SD) | 168.00 | 73.04% |
| Tartaric acid (powder) | 20.00 | 8.70% |
| Croscarmellose Sodium (Ac-Di-Sol) | 10.00 | 4.35% |

Part II - Wet granulation

| | | |
|---|---|---|
| Saxagliptin base (monohydrate) | 5.00 | 2.17% |
| Hypromellose (Pharmacoat 606) | 10.00 | 4.35% |
| pH 2.0 Acidic medium* | 20.00 | |

Part III - Mixing II

| | | |
|---|---|---|
| Croscarmellose Sodium (Ac-Di-Sol) | 10.00 | 4.35% |

Part IV - Mixing II

| | | |
|---|---|---|
| Magnesium stearate | 2.00 | 0.87% |

Part V - Cosmetic coating

| | | |
|---|---|---|
| Opadry II Pink (Polyvinyl alcohol based) | 5.00 | 2.17% |
| pH 2.0 Acidic Medium* | 28.00 | |
| Total weight | 230.00 | 100% |

*Evaporated during process

Further aspects and embodiments of the present invention are described with reference to the following numbered clauses:
1. A compressed solid dosage form comprising saxagliptin or a pharmaceutically acceptable salt thereof and at least one pharmaceutically acceptable excipient.
2. A dosage form according to clause 1 comprising at least one pharmaceutically acceptable excipient selected from the group consisting of filler, organic acid, disintegrant, lubricant, and binder, or a mixture thereof.
3. A dosage form according to clause 2 comprising at least one pharmaceutically acceptable excipient selected from the group consisting of filler, organic acid, disintegrant, and lubricant, or a mixture thereof.
4. A dosage form according to any of clauses 1-3 wherein the pharmaceutically acceptable excipient includes at least one organic acid.
5. A dosage form according to any of clauses 1-4 wherein the pharmaceutically acceptable excipient includes at least a filler, organic acid, disintegrant and lubricant.
6. A dosage form according to any of clauses 2-5 wherein the filler is selected from the group consisting of microcrystalline cellulose, sorbitol, dextrose, sucrose, mannitol, dibasic calcium phosphate, starch and mixtures thereof.
7. A dosage form according to clause 6 wherein the filler is selected from the group consisting of microcrystalline cellulose, sorbitol, dextrose, sucrose, mannitol, starch, and mixtures thereof.
8. A dosage form according to clause 7 wherein the filler is selected from the group consisting of microcrystalline cellulose, mannitol, and mixtures thereof.
9. A dosage form according to clause 8 wherein the filler is mannitol.
10. A dosage form according to any of clauses 2-9 wherein the filler is present in an amount of about 60 to about 90 wt %.
11. A dosage form according to clause 10 wherein the filler is present in an amount of about 65 to about 85 wt %.
12. A dosage form according to clause 11 wherein the filler is present in an amount of about 70 to about 82 wt %.
13. A dosage form according to clause 12 wherein the filler is present in an amount of about 72 to about 80 wt %.
14. A dosage form according to any of clauses 2-13 wherein the organic acid has a pKa1 greater than 3.
15. A dosage form according to of any of clauses 2-14 wherein the organic acid is a solid organic acid.
16. A dosage form according to any of clauses 2-15, wherein the organic acid has a solubility in water at 20-25° C. of 10 mg/ml or more.
17. A dosage form according to clause 16 wherein the organic acid has a solubility in water at 20-25° C. of 30 mg/ml or more.
18. A dosage form according to clause 17 wherein the organic acid has a solubility in water at 20-25° C. of 50 mg/ml or more.
19. A dosage form according to clause 18 wherein the organic acid has a solubility in water at 20-25° C. of 100 mg/ml or more.
20. A dosage form according to any of clauses 2-15 wherein the pharmaceutically acceptable acid is selected from the group consisting of tartaric acid, fumaric acid, succinic acid, citric acid, ascorbic acid and mixtures thereof.
21. A dosage form according to clause 20 wherein the organic acid is a solid organic acid, preferably selected from the group consisting of tartaric acid, succinic acid, citric acid, ascorbic acid and mixtures thereof.
22. A dosage form according to clause 21 wherein the organic acid is selected from the group consisting of tartaric acid, citric acid, and mixtures thereof.
23. A dosage form according to clause 22 wherein the organic acid is tartaric acid, preferably L-tartaric acid.
24. A dosage form according to any of clauses 2-23 wherein the organic acid is present in an weight excess relative to the saxagliptin or pharmaceutically acceptable salt thereof.
25. A dosage form according to clause 24 wherein the weight ratio of saxagliptin or pharmaceutically acceptable salt thereof to the organic acid is in the range of from 1:5 to 1:12.
26. A dosage form according to clause 25 wherein the weight ratio of saxagliptin or pharmaceutically acceptable salt thereof to the organic acid is in the range of from 1:6 to 1:10.
27. A dosage form according to clause 26 wherein the weight ratio of saxagliptin or pharmaceutically acceptable salt thereof to the organic acid is in the range of from 1:5 to 1:8.
28. A dosage form according to any of clauses 2-23 wherein the organic acid is present in an amount of about 5 to about 25 wt % based on the total weight of the composition.
29. A dosage form according to clause 28 wherein the organic acid is present in an amount of about 5 to about 15 wt % based on the total weight of the composition.
30. A dosage form according to clause 29 wherein the organic acid is present in an amount of about 6 to about 12 wt % based on the total weight of the composition.
31. A dosage form according to clause 30 wherein the organic acid is present in an amount of about 8 to about 10 wt % based on the total weight of the composition.
32. A dosage form according to any of clauses 2-31 wherein the disintegrant is selected from the group consisting of croscarmellose sodium, alginic acid, microcrystalline cellulose, crospovidone, polacrilin potassium, sodium starch glycolate, low-substituted hydroxypropyl cellulose, starch, and mixtures thereof.
33. A dosage form according to clause 32 wherein the disintegrant is selected from the group consisting of croscarmellose sodium, alginic acid, crospovidone, sodium starch glycolate, and mixtures thereof.
34. A dosage form according to clause 33 wherein the disintegrant is croscarmellose sodium.
35. A dosage form according to any of clauses 2-34 wherein the disintegrant is present in an amount of about 4 to about 15 wt %.
36. A dosage form according to clause 35 wherein the disintegrant is present in an amount of about 5 to about 13 wt %.
37. A dosage form according to clause 36 wherein the disintegrant is present in an amount of about 7 to about 11 wt %.
38. A dosage form according to any of clauses 2-37 wherein the lubricant is selected from the group consisting of magnesium stearate, calcium stearate, glyceryl behenate, sodium stearyl fumarate, stearic acid, talc, zinc stearate, and mixtures thereof.
39. A dosage form according to clause 38 wherein the lubricant is selected from the group consisting of magnesium stearate, glyceryl behenate, sodium stearyl fumarate, talc and mixtures thereof.
40. A dosage form according to clause 39 wherein the lubricant is selected from the group consisting of magnesium stearate, talc or mixtures thereof.
41. A dosage form according to clause 40 wherein the lubricant is magnesium stearate.
42. A dosage form according to any of clauses 2-41 wherein the lubricant is present in an amount of about 0.3% to about 3 wt %.
43. A dosage form according to clause 42 wherein the lubricant is present in an amount of about 0.5 to about 2 wt %.
44. A dosage form according to clause 43 wherein the lubricant is present in an amount of about 0.4 to about 1.5 wt %.
45. A dosage form according to any of clauses 1-44 wherein the pharmaceutically acceptable excipient includes a binder.
46. A dosage form according to clause 45 wherein the binder is selected from the group consisting of polyvinyl pyrrolidone, hydroxypropyl cellulose, low-substituted hydroxypropyl cellulose, hydroxypropyl methyl cellulose, polyvinyl alcohol, starch and mixtures thereof, preferably hydroxypropyl methyl cellulose.
47. A dosage form according to clause 46 wherein the binder is selected from the group consisting of polyvinyl pyrrolidone, hydroxypropyl cellulose, low-substituted hydroxypropyl cellulose, hydroxypropyl methyl cellulose, polyvinyl alcohol and mixtures thereof.
48. A dosage form according to clause 47 wherein the binder is present in an amount of about 2 to about 6 wt %.
49. The dosage form of clause 48 wherein the binder is present in an amount of about 3 to about 5 wt %.
50. The dosage form of any preceding clause wherein the saxagliptin or pharmaceutically acceptable salt thereof is present in an amount of about 1.0 to about 5 wt %.
51. The dosage form of clause 50 wherein the saxagliptin or pharmaceutically acceptable salt thereof is present in an amount of about 1.2 to about 3.0 wt %.
52. The dosage form of clause 51 wherein the saxagliptin or pharmaceutically acceptable salt thereof is present in an amount of about 1.2 to about 2.3 wt %.
53. A dosage form according to any of clauses 1-52 wherein the saxagliptin is in the form of a pharmaceutically acceptable salt.
54. A dosage form according to clause 53 wherein the saxagliptin is in the form of a salt selected from hydrochloride, hydrobromide, phosphate or solvates and hydrates thereof.
55. A dosage form according to clause 54 wherein the saxagliptin is in the form of its hydrochloride or hydrobromide salt, or solvates and hydrates thereof.
56. A dosage form according to clause 55 wherein the saxagliptin is in the form of its hydrochloride salt or solvates and hydrates thereof.
57. A dosage form according to any of clauses 1-56 containing less than 0.5 wt % of saxagliptin cyclic amidine 30 days after manufacture.
58. A dosage form according to clause 57 containing 0.4 wt % or less of saxagliptin cyclic amidine.
59. A dosage form according to clause 58 containing 0.35 wt % or less of saxagliptin cyclic amidine.
60. A dosage form according to clause 59 containing 0.3 wt % or less of saxagliptin cyclic amidine.
61. A dosage form according to any preceding clause comprising saxagliptin or a pharmaceutically acceptable salt thereof, preferably saxagliptin hydrochloride, and the following excipients: mannitol, tartaric acid, croscarmellose sodium, magnesium stearate, and optionally hydroxypropyl methylcellulose.
62. A dosage form according to any preceding clause wherein the dosage form contains a cosmetic, enteric, or extended release coating.
63. A dosage form according to any preceding clause having a hardness of 7 to 25 Strong-Cobb units (SCU).
64. A dosage form according to clause 63 having a hardness of 7-20 SCU.
65. A dosage form according to clause 64 having a hardness of 8-18 SCU.
66. A dosage form according to clause 1 prepared by compression of a mixture of saxagliptin or a pharmaceutically acceptable salt thereof and a least one pharmaceutically acceptable excipient.
67. A dosage form according to clause 66 wherein the excipient is as defined in any of clauses 2-49.
68. A dosage form according to clause 67 wherein the excipient comprises the following: mannitol, tartaric acid, croscarmellose sodium, magnesium stearate, and optionally hydroxypropyl methylcellulose.
69. A dosage form according to any of clauses 66 to 68, wherein the saxagliptin or pharmaceutically acceptable salt thereof is as defined in any of clauses 50-56.
70. A dosage form according to any of clauses 66 to 69 containing less than 0.5 wt % of saxagliptin cyclic amidine 30 days after manufacture.
71. A dosage form according to clause 70 containing 0.4 wt % or less of saxagliptin cyclic amidine or pharmaceutically acceptable salts thereof.
72. A dosage form according to clause 71 containing 0.35 wt % or less of saxagliptin cyclic amidine.
73. A dosage form according to clause 72 containing 0.3 wt % or less of saxagliptin cyclic amidine.
74. A dosage form according to any of clauses 66 to 73 having a hardness of 7 to 25 Strong-Cobb units (SCU).
75. A dosage form according to clause 74 having a hardness of 7-20 SCU.
76. A dosage form according to clause 75 having a hardness of 8-18 SCU.
77. A dosage form according to any preceding clause containing 2.5 mg or 5 mg equivalent of saxagliptin free base.
78. A process for preparing the dosage form of any of clauses 1-77 comprising:
   (a) providing a mixture of the saxagliptin or a pharmaceutically acceptable salt thereof and the at least one pharmaceutically acceptable excipient; and
   (b) compressing the mixture.
79. A process according to clause 78 wherein the mixture in step (a) is prepared by wet granulation or dry granulation.
80. A process according to clause 78 or clause 79 wherein the mixture in step (a) is prepared by wet granulation.
81. A process according to clause 80 wherein the saxagliptin is provided as a free base which is contacted with a solution of a suitable pharmaceutically acceptable acid to form the corresponding pharmaceutically acceptable salt of saxagliptin.
82. A process according to clause 81 wherein the solution of a pharmaceutically acceptable acid is employed as the wet granulation medium.
83. A process according to clause 82 wherein the pharmaceutically acceptable acid is hydrochloric acid, hydrobromic acid, or phosphoric acid, thereby forming the corresponding hydrochloride, hydrobromide, or phosphate salt of saxagliptin.
84. A process according to clause 83 wherein the pharmaceutically acceptable acid is hydrochloric acid, thereby forming saxagliptin hydrochloride.
85. A process according to any of clauses 79-84 wherein the wet granulated mixture is subjected to a drying step.
86. A process according to clause 85 wherein the dried mixture is milled.
87. A process according to any of clauses 78 to 86 wherein the compressed mixture is coated with a cosmetic, enteric or extended release coating.
88. A process according to clause 87 wherein the cosmetic coating includes a coating polymer, preferably selected from the group consisting of polyvinyl pyrrolidone, hydroxypropyl cellulose, hydroxypropyl methyl cellulose, polyvinyl alcohol and mixtures thereof, and preferably wherein the cosmetic coating is selected from the group consisting of polyvinyl pyrrolidone, polyvinyl alcohol or a mixture thereof, and more preferably polyvinyl alcohol.
89. A dosage form obtainable by a process according to any of clauses 78 to 88.
90. A pharmaceutical dosage form comprising saxagliptin or a pharmaceutically acceptable salt thereof and at least one solid pharmaceutically acceptable organic acid.
91. A dosage form according to clause 90 wherein the organic acid has a pKa1 greater than 3.
92. A dosage form according to clause 90 or clause 91 wherein the organic acid has a solubility in water at 20-25° C. of 10 mg/ml or more.
93. A dosage form according to clause 92 wherein the organic acid has a solubility in water at 20-25° C. of 30 mg/ml or more.
94. A dosage form according to clause 93 wherein the organic acid has a solubility in water at 20-25° C. of 50 mg/ml or more.
95. A dosage form according to clause 94 wherein the organic acid has a solubility in water at 20-25° C. of 100 mg/ml or more.
96. A dosage form according to any of clauses 90 to 95 wherein the pharmaceutically acceptable organic acid is a solid organic acid, preferably selected from the group consisting of tartaric acid, fumaric acid, succinic acid, citric acid, ascorbic acid and mixtures thereof.
97. A dosage form according to clause 96 wherein the organic acid is selected from the group consisting of tartaric acid, citric acid, and mixtures thereof.
98. A dosage form according to clause 97 wherein the organic acid is tartaric acid, preferably L-tartaric acid.
99. A dosage form according to any of clauses 90 to 98 wherein the organic acid is present in an excess relative to the saxagliptin or pharmaceutically acceptable salt thereof.
100. A dosage form according to clause 99 wherein the weight ratio of saxagliptin or pharmaceutically acceptable salt thereof to the organic acid is in the range of from 1:5 to 1:12.
101. A dosage form according to clause 100 wherein the weight ratio of saxagliptin or pharmaceutically acceptable salt thereof to the organic acid is in the range of from 1:6 to 1:10.
102. A dosage form according to clause 101 wherein the weight ratio of saxagliptin or pharmaceutically acceptable salt thereof to the organic acid is in the range of from 1:5 to 1:8.
103. A dosage form according to any of clauses 90 to 102 wherein the organic acid is present in an amount of about 5 to about 25 wt % based on the total weight of the composition.
104. A dosage form according to clause 103 wherein the organic acid is present in an amount of about 5 to about 15 wt % based on the total weight of the composition.
105. A dosage form according to clause 104 wherein the organic acid is present in an amount of about 6 to about 12 wt % based on the total weight of the composition.
106. A dosage form according to clause 105 wherein the organic acid is present in an amount of about 8 to about 10 wt % based on the total weight of the composition.
107. A dosage form according to any of clauses 90 to 106 further comprising at least one pharmaceutically acceptable excipient.
108. A dosage form according to clause 107 where the pharmaceutically acceptable excipient is selected from the group consisting of filler, disintegrant, lubricant, and binder, or a mixture thereof.
109. A dosage form according to clause 108 wherein the pharmaceutically acceptable excipient includes at least a filler, disintegrant and lubricant.
110. A dosage form according to any of clauses 90 to 109 wherein the pharmaceutically acceptable excipient is as defined in any of clauses 6-13 and 32-49.
111. A dosage form according to any of clauses 90 to 110 wherein the saxagliptin is as defined in any of clauses 50 to 56.
112. A dosage form according to any of clauses 90 to 111 wherein the dosage form comprises saxagliptin or a pharmaceutically acceptable salt thereof, and the following excipients: mannitol, tartaric acid, croscarmellose sodium, magnesium stearate, and optionally hydroxypropyl methylcellulose.
113. A dosage form according to any of clauses 90 to 112 containing 2.5 mg or 5 mg equivalent of saxagliptin free base.
114. Use of a solid pharmaceutically acceptable organic acid to stabilize a pharmaceutical dosage form comprising saxagliptin or a pharmaceutically acceptable salt thereof.
115. Use according to clause 114 wherein the pharmaceutically acceptable organic acid is as defined in any of clauses 91 to 106.
116. Use according to clause 114 or clause 115, wherein the pharmaceutical dosage form comprises saxagliptin or a pharmaceutically acceptable salt thereof and a pharmaceutically acceptable excipient as defined in any of clauses 6-13 and 32-49.
117. Use according to any of clauses 114 to 116, wherein the dosage form comprises saxagliptin or a pharmaceutically acceptable salt thereof, and the following excipients: mannitol, tartaric acid, croscarmellose sodium, magnesium stearate, and optionally hydroxypropyl methylcellulose.
118. Use according to any of clauses 114 to 117 wherein the saxagliptin is as defined in any of clauses 50 to 56.
119. Use according to any of clauses 114 to 118 wherein the dosage form contains 2.5 mg or 5 mg equivalent of saxagliptin free base.

1. A compressed solid dosage form comprising saxagliptin or a pharmaceutically acceptable salt thereof and at least one pharmaceutically acceptable excipient.

2. A dosage form according to claim 1, wherein the at least one pharmaceutically acceptable excipient comprises at least one organic acid, filler, disintegrant, lubricant, binder, or a mixture thereof.

3. A dosage form according to claim 2, wherein the at least one pharmaceutically acceptable excipient comprises at least one organic acid, filler, disintegrant, lubricant, or a mixture thereof.

4. A dosage form according to claim 1, wherein the at least one pharmaceutical comprises at least one organic acid.

5. A dosage form according to claim 4, wherein the pharmaceutically acceptable excipient further comprises a filler, disintegrant and lubricant.

6. A dosage form according to claim 2, wherein the filler is microcrystalline cellulose, sorbitol, dextrose, sucrose, mannitol, dibasic calcium phosphate, starch, or mixtures thereof.

7. A dosage form according to claim 6, wherein the filler is microcrystalline cellulose, sorbitol, dextrose, sucrose, mannitol, or a mixture thereof.

8. A dosage form according to claim 7, wherein the filler is mannitol.

9. A dosage form according to claim 2, wherein the filler is present in an amount of about 60 to about 90 wt %.

10. A dosage form according to claim 9, wherein the filler is present in an amount of about 70 to about 82 wt %.

11. A dosage form according to claim 4, wherein the at least one organic acid comprises at least one organic acid having a pKa or pKa1 of 5 or less.

12. A dosage form according to claims 11, wherein the at least one organic acid comprises at least one organic acid having a pKa1 ranging from 2.5 to 5.

13. A dosage form according to claim 12, wherein the at least one organic acid comprises at least one organic acid having a pKa1 ranging from about 3 to less than 5.

14. A dosage form according to claim 4, wherein the at least one organic acid comprises at least one organic acid having a pKa1 greater than 3.

15. A dosage form according to claim 4, wherein the at least one organic acid is at least one solid organic acid.

16. A dosage form according to claim 4, wherein the at least one organic acid has a solubility in water at 20-25° C. of 10 mg/ml or more.

17. A dosage form according to claim 16 wherein the at least one organic acid has a solubility in water at 20-25° C. of 30 mg/ml or more.

18. A dosage form according to claim 17 wherein the at least one organic acid has a solubility in water at 20-25° C. of 50 mg/ml or more.

19. A dosage form according to claim 18 wherein the at least one organic acid has a solubility in water at 20-25° C. of 100 mg/ml or more.

20. A dosage form according to claim 4, wherein the at least one organic acid is tartaric acid, fumaric acid, succinic acid, citric acid, ascorbic acid, or a mixture thereof.

21. A dosage form according to claim 20, wherein the tartaric acid, succinic acid, citric acid, ascorbic acid, or a mixture thereof is or are solid.

22. A dosage form according to claim 21, wherein the at least one organic acid is tartaric acid, citric acid, or a mixture thereof.

23. A dosage form according to claim 22, wherein the at least one organic acid is tartaric acid, preferably L-tartaric acid.

24. A dosage form according to claim 4, wherein the at least one organic acid is present in an weight excess relative to the saxagliptin or pharmaceutically acceptable salt thereof.

25. A dosage form according to claim 24, wherein the weight ratio of saxagliptin or pharmaceutically acceptable salt thereof to the at least one organic acid is in the range of from 1:5 to 1:12.

26. A dosage form according to claim 4, wherein the at least one organic acid is present in an weight not in excess relative to the saxagliptin or pharmaceutically acceptable salt thereof.

27. A dosage form according to claim 26, wherein the dosage form comprises a pharmaceutically acceptable salt of saxagliptin, and wherein the at least one organic acid is present in an weight less than the weight of the pharmaceutically acceptable salt of saxagliptin.

28. A dosage form according to claim 4, wherein the at least one organic acid is present in an amount of about 5 to about 25 wt % based on the total weight of the dosage form.

29. A dosage form according to claim 28 wherein the at least one organic acid is present in an amount of about 5 to about 15 wt % based on the total weight of the dosage form.

30. A dosage form according to claim 29, wherein the at least one organic acid is present in an amount of about 6 to about 12 wt % based on the total weight of the dosage form.

31. A dosage form according to claim 30, wherein the at least one organic acid is present in an amount of about 8 to about 10 wt % based on the total weight of the dosage form.

32. A dosage form according to claim 2, wherein the disintegrant is croscarmellose sodium, alginic acid, microcrystalline cellulose, crospovidone, polacrilin potassium, sodium starch glycolate, low-substituted hydroxypropyl cellulose, starch, or a mixture thereof.

33. A dosage form according to claim 32, wherein the disintegrant is croscarmellose sodium, alginic acid, crospovidone, sodium starch glycolate, or a mixture thereof.

34. A dosage form according to claim 33, wherein the disintegrant is croscarmellose sodium.

35. A dosage form according to claim 2, wherein the disintegrant is present in an amount of about 4 to about 15 wt %.

36. A dosage form according to claim 35, wherein the disintegrant is present in an amount of about 5 to about 13 wt %.

37. A dosage form according to claim 36, wherein the disintegrant is present in an amount of about 7 to about 11 wt %.

38. A dosage form according to claim 2, wherein the lubricant is magnesium stearate, calcium stearate, glyceryl behenate, sodium stearyl fumarate, stearic acid, talc, zinc stearate, or a mixture thereof.

39. A dosage form according to claim 38, wherein the lubricant is magnesium stearate, glyceryl behenate, sodium stearyl fumarate, talc or a mixture thereof.

40. A dosage form according to claim 39, wherein the lubricant is magnesium stearate, talc or a mixture thereof.

41. A dosage form according to claim 40, wherein the lubricant is magnesium stearate.

42. A dosage form according to claim 2, wherein the lubricant is present in an amount of about 0.3% to about 3 wt %.

43. A dosage form according to claim 42, wherein the lubricant is present in an amount of about 0.5 to about 2 wt %.

44. A dosage form according to claim 43, wherein the lubricant is present in an amount of about 0.4 to about 1.5 wt %.

45. A dosage form according to claim 2, wherein the at least one pharmaceutically acceptable excipient includes binder.

46. A dosage form according to claim 45, wherein the binder is polyvinyl pyrrolidone, hydroxypropyl cellulose, low-substituted hydroxypropyl cellulose, hydroxypropyl methyl cellulose, polyvinyl alcohol, starch or a mixture thereof, preferably hydroxypropyl methyl cellulose.

47. A dosage form according to claim 46, wherein the binder is polyvinyl pyrrolidone, hydroxypropyl cellulose, low-substituted hydroxypropyl cellulose, hydroxypropyl methyl cellulose, polyvinyl alcohol, or a mixture thereof.

48. A dosage form according to claim 47, wherein the binder is present in an amount of about 2 to about 6 wt %.

49. The dosage form of claim 48, wherein the binder is present in an amount of about 3 to about 5 wt %.

50. The dosage form of claim 1, wherein the saxagliptin or pharmaceutically acceptable salt thereof is present in an amount of about 1.0 to about 5 wt %.

51. The dosage form of claim 50, wherein the saxagliptin or pharmaceutically acceptable salt thereof is present in an amount of about 1.2 to about 3.0 wt %.

52. The dosage form of claim 51, wherein the saxagliptin or pharmaceutically acceptable salt thereof is present in an amount of about 1.2 to about 2.3 wt %.

53. A dosage form according to claim 1, wherein the saxagliptin is in the form of a pharmaceutically acceptable salt.

54. A dosage form according to claim 53, wherein the saxagliptin is in the form of a salt selected from hydrochloride, hydrobromide, phosphate or solvates and hydrates thereof.

55. A dosage form according to claim 54, wherein the saxagliptin is in the form of its hydrochloride or hydrobromide salt, or solvates and hydrates thereof.

56. A dosage form according to claim 55, wherein the saxagliptin is in the form of its hydrochloride salt or solvates and hydrates thereof.

57. A dosage form according to claim 1 containing less than 0.5 wt % of saxagliptin cyclic amidine 30 days after manufacture following storage at a temperature of between 15-29° C., at a relative humidity of not more than 67%.

58. A dosage form according to claim 57 containing 0.4 wt % or less of saxagliptin cyclic amidine.

59. A dosage form according to claim 58 containing 0.35 wt % or less of saxagliptin cyclic amidine.

60. A dosage form according to claim 59 containing 0.3 wt % or less of saxagliptin cyclic amidine.

61. A dosage form according to claim 1 comprising saxagliptin or a pharmaceutically acceptable salt thereof, preferably saxagliptin hydrochloride, and the following excipients: mannitol, tartaric acid, croscarmellose sodium, magnesium stearate, and optionally hydroxypropyl methylcellulose.

62. A dosage form according to claim 1 wherein the dosage form contains a cosmetic, enteric, or extended release coating.

63. A dosage form according to claim 1 having a hardness of 7 to 25 Strong-Cobb units (SCU).

64. A dosage form according to claim 63 having a hardness of 7-20 SCU.

65. A dosage form according to claim 64 having a hardness of 8-18 SCU.

66. A dosage form according to claim 1 prepared by compression of a mixture of saxagliptin or a pharmaceutically acceptable salt thereof and a least one pharmaceutically acceptable excipient.

67. A dosage form according to claim 66, wherein the at least one pharmaceutically acceptable excipient is as defined in any one of claims 2-49.

68. A dosage form according to claim 67, wherein the at least one pharmaceutically acceptable excipient comprises the following: mannitol, tartaric acid, croscarmellose sodium, magnesium stearate, and optionally hydroxypropyl methylcellulose.

69. A dosage form according to claim 66, wherein the saxagliptin or pharmaceutically acceptable salt thereof is present in an amount of about 1.0 to about 5 wt %.

70. A dosage form according to claim 66 containing less than 0.5 wt % of saxagliptin cyclic amidine 30 days after manufacture following storage at a temperature of between 15-29° C., at a relative humidity of not more than 67%.

71. A dosage form according to claim 70 containing 0.4 wt % or less of saxagliptin cyclic amidine or pharmaceutically acceptable salts thereof.

72. A dosage form according to claim 71 containing 0.35 wt % or less of saxagliptin cyclic amidine.

73. A dosage form according to claim 72 containing 0.3 wt % or less of saxagliptin cyclic amidine.

74. A dosage form according to claim 66 having a hardness of 7 to 25 Strong-Cobb units (SCU).

75. A dosage form according to claim 74 having a hardness of 7-20 SCU.

76. A dosage form according to claim 75 having a hardness of 8-18 SCU.

77. A dosage form according to claim 1 containing 2.5 mg or 5 mg equivalent of saxagliptin free base.

78. A process for preparing the dosage form of claim 1 comprising:
(a) providing a mixture of the saxagliptin or a pharmaceutically acceptable salt thereof and the at least one pharmaceutically acceptable excipient; and
(b) compressing the mixture.

79. A process according to claim 78, wherein the mixture in step (a) is prepared by wet granulation or dry granulation.

80. A process according to claim 78 or claim 79, wherein the mixture in step (a) is prepared by wet granulation.

81. A process according to claim 80, wherein the saxagliptin is provided as a free base which is contacted with a solution of a suitable pharmaceutically acceptable acid to form the corresponding pharmaceutically acceptable salt of saxagliptin.

82. A process according to claim 81, wherein the solution of the pharmaceutically acceptable acid is employed as the wet granulation medium.

83. A process according to claim 82, wherein the pharmaceutically acceptable acid is hydrochloric acid, hydrobromic acid, or phosphoric acid, thereby forming the corresponding hydrochloride, hydrobromide, or phosphate salt of saxagliptin.

84. A process according to claim 83, wherein the pharmaceutically acceptable acid is hydrochloric acid, thereby forming saxagliptin hydrochloride.

85. A process according to claim 79, wherein the wet granulated mixture is subjected to drying.

86. A process according to claim 85, wherein the mixture subjected to drying is milled.

87. A process according to claim 78, wherein the mixture in step (a) is subjected to direct compression in step (b).

88. A process according to claim 78, wherein the compressed mixture is coated with a cosmetic, enteric or extended release coating.

89. A process according to claim 88, wherein the compressed mixture is coated with a cosmetic coating and wherein the cosmetic coating includes a coating polymer, preferably selected from polyvinyl pyrrolidone, hydroxypropyl cellulose, hydroxypropyl methyl cellulose, polyvinyl alcohol and mixtures thereof, and preferably wherein the cosmetic coating is selected from polyvinyl pyrrolidone, polyvinyl alcohol or a mixture thereof, and more preferably polyvinyl alcohol.

90. A dosage form obtainable by a process according to claim 78.

91. A method for stabilizing a pharmaceutical dosage form comprising saxagliptin or a pharmaceutically acceptable salt thereof, the method comprising mixing the saxagliptin or a pharmaceutically acceptable salt thereof with at least one pharmaceutically acceptable excipient comprising at least one pharmaceutically acceptable organic acid.

92. A method according to claim 91, wherein the at least one pharmaceutically acceptable organic acid is solid having a pKa or pKa1 of 5 or less.

93. A method according to claim 91, wherein the at least one pharmaceutically acceptable excipient further comprises a filler, disintegrant, lubricant, binder or a mixture thereof.

94. A method according to claim 91, wherein the at least one pharmaceutically acceptable excipient comprises mannitol, tartaric acid, croscarmellose sodium, magnesium stearate, and optionally hydroxypropyl methylcellulose.

95. A method according to claim 91, wherein the saxagliptin is present in an amount of about 1.0 to about 5 wt %.

96. A method according to claim 91, wherein the dosage form prepared by the method contains 2.5 mg or 5 mg equivalent of saxagliptin free base.
